(19) United States
(12) Patent Application Publication
(10) Pub. No.: US 2017/0349534 A1
(43) Pub. Date: Dec. 7, 2017

(54) A NOVEL PROCESS FOR CHIRAL RESOLUTION OF HIGHLY PURE (6R)-6-(DIMETHYLAMINO)-4,4-DIPHENYL-HEPTANONE FROM RACEMIC METHADONE HYDROCHLORIDE

(71) Applicant: HARMAN FINOCHEM LIMITED, Mumbai (IN)

(72) Inventors: Vijay Trimbak KADAM, Aurangabad (IN); Dhananjay Uddavrao EDAKI, Aurangabad (IN); Nagaraju ORUGANTI, Aurangabad (IN)

(21) Appl. No.: 15/539,085

(22) PCT Filed: Apr. 30, 2015
(86) PCT No.: PCT/IN2015/000190

§ 371 Date: Jun. 22, 2017

(30) Foreign Application Priority Data
Dec. 22, 2014 (IN) 4118/MUM/2014

(51) Int. Cl. C07C 221/00; C07C 225/16

(52) U.S. Cl. CPC C07C 221/00; C07C 225/16; C07B 2200/07

(57) ABSTRACT

The present invention discloses an improved process for preparing pure (6R)-6-(dimethylamino)-4,4-diphenyl-3-heptanone and its hydrochloride salt.

### FIELD OF INVENTION

The present invention relates to a novel, cost-effective, efficient and advantageous process for preparing (6R)-6-(dimethylamino)-4,4-diphenyl-3-heptanone.

### BACKGROUND OF INVENTION

(6R)-6-(dimethylamino)-4,4-diphenyl-3-heptanone, or R-(−)-methadone having Formula I, is anactive enantiomer of methadone useful in pain management and opioid maintenance therapy.

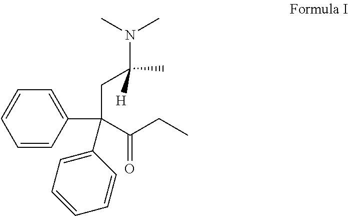 [SMILES: CCC(=O)C(C[C@@H](C)N(C)C)(c1ccccc1)c1ccccc1]

Methadone is an analgesic compound possessing various therapeutic properties of morphine and is well known in the medical field.

Methadone possesses one asymmetric carbon atom and is thus capable of existing in dextro, levo and racemic form. It is also well known that one of the optical isomers may have properties rendering it many times more useful than the other optical isomers. In case of dl-methadone, levo isomer is having marked analgesic activity as compared to dextro isomer.

Thus the isolation of these pure forms is desirable.

Prior art discloses various methods to prepare methadone. U.S. Pat No. 2,601,323 discloses process for preparing 4,4-diphenyl-6-dimethylamino-heptanone-3. J. Amer. Chem. Soc., 1947, 70 (12), 4194-97; J. Org. Chem. 1948, 13, 191 describes process for preparing optically active methadones. Many chemical compounds are obtained naturally or synthesized in the form of racemic mixture of optical isomers. There are various procedures for resolution of the racemic mixture. One known method is placing seed of one of the optical isomers in a solution containing the racemic composition and allowing crystal growth on this seed from the corresponding optical isomer in the solution. Another method is physically separating optical isomers from a racemic mixture by using special and sophisticated apparatus which are expensive. Yet another method reported is enzymatic resolution of the optical isomers. The above described procedures for resolution of the racemic mixture are unsatisfactory and expensive. Thus there is an urgent need to develop industrially viable and workable, economical and time-saving process for resolution of the racemic mixture.

U.S. Pat No. 6,143,933 discloses enzymatic resolution of 1-dialkylamino-2-propanol in the presence of ester so as to produce S-1-dialkylamino-2-propanol and R-ester of 1-dialkylamino-2-propanol, which further involves the conversion of one or both of the S-1-dialkylamino-2-propanol and/or R-ester of 1-dialkylamino-2-propanol to yield S(+)-methadone and/or R(−)-methadone respectively. (Scheme 1)

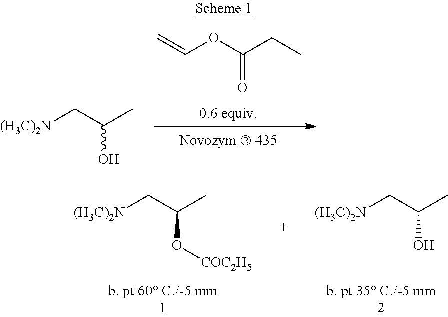

The enzymes employed in the prior art for preparing optically active methadones are very expensive thereby rendering the process non-economical.

Thus there exists an urgent need in isolating pure form of levo isomer of methadone by employing an efficient and cost-effective process.

Therefore, the object of the present invention is to provide a cost-effective method for resolving racemic mixtures of optical isomers of dl-methadoneto obtain highly pure (6R)-6-(dimethylamino)-4,4-diphenyl-3-heptanone with higher enantiomeric purity.

The resolving agent used in the present invention is cheap as compared to other resolving agents described in prior art.

### SUMMARY OF THE INVENTION

The present invention provides a novel process for preparing optically active (6R)-6-(dimethylamino)-4,4-diphenyl-3-heptanone (Formula I) with high enantiomeric purity.

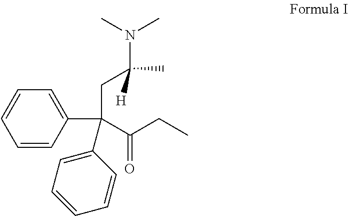 [SMILES: CCC(=O)C(C[C@@H](C)N(C)C)(c1ccccc1)c1ccccc1]

In one aspect, present invention provides process for preparing (+)-N-protected-L-glutamic acid (Formula II) from L-glutamic acid comprising

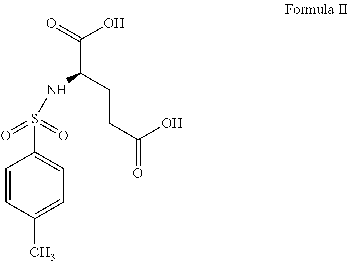 [SMILES: Cc1ccc(S(=O)(=O)N[C@H](CCC(=O)O)C(=O)O)cc1]

Reacting L-glutamic acid with N-protected group in presence of base at 70-75° C. at basic pH less than 9

Acidifying the reaction mass by adjusting pH 1.5-2.0

Extracting organic layer

Isolating (+)-N-protected-L-glutamic acid from L-glutamic acid

In another aspect, present invention provides preparation of (RS)-6-(dimethylamino)-4,4-diphenylheptan-3-one free base (Formula III) from (RS)-6-(dimethylamino)-4,4-diphenylheptan-3-one hydrochloride salt comprising

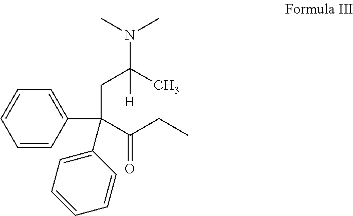 [SMILES: CCC(=O)C(CC(C)N(C)C)(c1ccccc1)c1ccccc1]

Basifying (RS)-6-(dimethylamino)-4,4-diphenylheptan-3-one hydrochloride salt by maintaining pH 8-12 using liquor ammonia at 25-30° C. and

Isolating (RS)-6-(dimethylamino)-4,4-diphenylheptan-3-one.

Yet another aspect of the present invention provides a process for resolution of the racemic mixture of (RS)-methadone by chemical reaction to get pure (6R)-6-(dimethylamino)-4,4-diphenyl-3-heptanone free base (Formula I) which comprises:

Reacting (RS)-6-(Dimethylamino)-4,4-diphenylheptan-3-one with(+)-N-protected-L-glutamic acid in presence of one or more organic solvents;

Isolating (6R)-6-(dimethylamino)-4,4-diphenyl-3-heptanone(+)-N-protected-L-glutamate salt;

Basifying (6R)-6-(dimethylamino)-4,4-diphenyl-3-heptanone(+)-N-protected-L-glutamate salt by adjusting pH 8-12 and

Isolating chirally pure (6R)-6-(dimethylamino)-4,4-diphenyl-3-heptanone free base

In another aspect, the present invention discloses process for preparing (6R)-6-(dimethylamino)-4,4-diphenyl-3-heptanone hydrochloride salt (Formula IV) from (6R)-6-(dimethylamino)-4,4-diphenyl-3-heptanone free base comprising

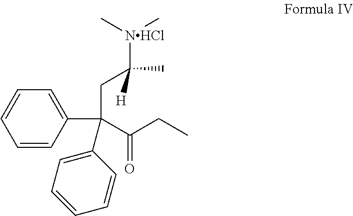

Reacting (6R)-6-(dimethylamino)-4,4-diphenyl-3-heptanone free base with alcoholic hydrochloride in presence of alcoholic solvent by adjusting pH 1-1.5 at 25-30° C.;

Treating with charcoal and

Isolating (6R)-6-(dimethylamino)-4,4-diphenyl-3-heptanone hydrochloride salt.

### DETAILED DESCRIPTION OF THE INVENTION

The invention will now be described in detail in connection with certain preferred and optional embodiments, so that various aspects thereof may be more fully understood and appreciated.

The present invention provides a novel process for preparing optically active (6R)-6-(dimethylamino)-4,4-diphenyl-3-heptanone (Formula I) with high enantiomeric purity.

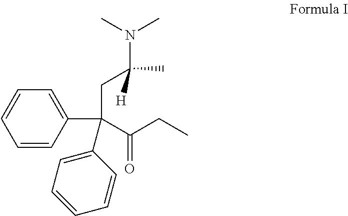 [SMILES: CCC(=O)C(C[C@@H](C)N(C)C)(c1ccccc1)c1ccccc1]

Formula I

According to one embodiment, present invention provides a process for preparing (+)-N-protected-L-glutamic acid from L-glutamic acid comprising:

Reacting L-glutamic acid with N-protected group in presence of base at 70-75° C. a basic pH less than 9;

Acidifying the reaction mass by adjusting pH 1.5-2.0;

Extracting organic layer and

Isolating (+)-N-protected-L-glutamic acid from L-glutamic acid.

The process for preparing (+)-N-tosyl-L-glutamic acid is depicted in the following reaction Scheme I.

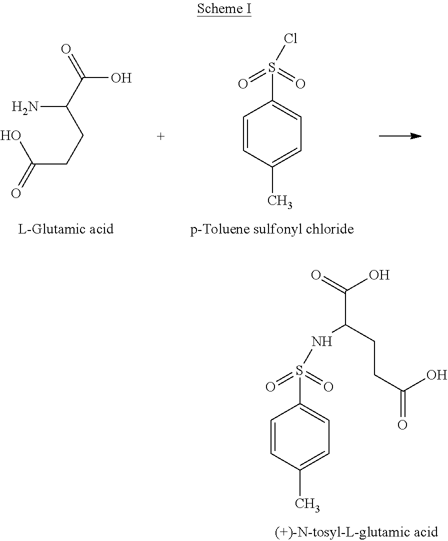 [SMILES: NC(CCC(=O)O)C(=O)O ; Cc1ccc(S(=O)(=O)Cl)cc1 ; Cc1ccc(S(=O)(=O)NC(CCC(=O)O)C(=O)O)cc1]

The N-protected group is preferably selected from messyl chloride, tosyl chloride, acetyl chloride. The term protected refers to messyl, tossyl, acetyl groups.

The base is selected from inorganic base. The inorganic base is preferably selected from carbonate salts and hydroxides of alkali and alkaline earth metals like potassium carbonate, potassium bicarbonate, sodium carbonate, sodium bicarbonate, cesium carbonate, calcium carbonate & sodium methoxide, sodium hydroxide, potassium hydroxide, calcium hydroxide, barium hydroxide, cesium hydroxide and lithium hydroxide.

Similar process as depicted in Scheme-I can be carried out for other protecting groups such as methane sulfonyl chloride, acetyl chloride.

In another aspect, present invention involves preparation of (RS)-6-(dimethylamino)-4,4-diphenylheptan-3-one free base from (RS)-6-(dimethylamino)-4,4-diphenylheptan-3-one hydrochloride salt which comprises:

Basifying (RS)-6-(dimethylamino)-4,4-diphenylheptan-3-one hydrochloride salt by maintaining pH between 8-12 using liquor ammonia at 25-30° C. and

Isolating (RS)-6-(dimethylamino)-4,4-diphenylheptan-3-one.

The reaction scheme for said process is given in Scheme II

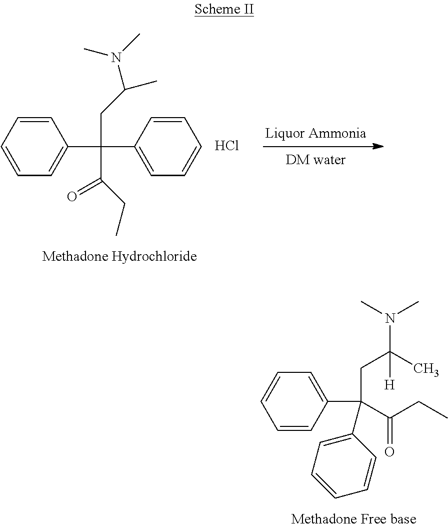 [SMILES: CCC(=O)C(CC(C)N(C)C)(c1ccccc1)c1ccccc1 ; Cl ; CCC(=O)C(CC(C)N(C)C)(c1ccccc1)c1ccccc1]

“Levorotation” refers to the properties of rotating plane polarized light. Light exhibits levorotation if the light rotates counterclockwise as it approaches the observer.

In one embodiment, the present invention provides a process for resolution of the racemic mixture of (RS)-methadone by chemical reaction to resolve pure (6R)-6-(dimethylamino)-4,4-diphenyl-3-heptanone which comprises:

Reacting (RS)-6-(Dimethylamino)-4,4-diphenylheptan-3-one with (+)-N-protected-L-glutamic acid in presence of organic solvent or mixture of two or more organic solvents;

Isolating (6R)-6-(dimethylamino)-4,4-diphenyl-3-heptanone-(+)-N-protected glutamate salt (Formula V);

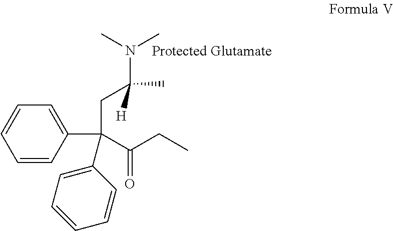

Basifying (6R)-6-(dimethylamino)-4,4-diphenyl-3-heptanone-(+)-N-protected glutamate salt by adjusting pH 8.5-9.5 and

Isolating (6R)-6-(dimethylamino)-4,4-diphenyl-3-heptanone free base.

In above said process the organic solvent is a ketone. The ketone is preferably selected from the group consisting of acetone, methyl ethyl ketone, diethyl ketone, methyl-tert-butyl ketone and isopropyl ketone. The said reaction is carried out at temperature of 40-60° C. The basification reaction is performed by employing the base selected from carbonate salts and hydroxides of alkali and alkaline earth metals like potassium carbonate, potassium bicarbonate, sodium carbonate, sodium bicarbonate, cesium carbonate, calcium carbonate & sodium methoxide, sodium hydroxide, potassium hydroxide, calcium hydroxide, barium hydroxide, cesium hydroxide and lithium hydroxide.

Accordingly, in yet another embodiment, the invention encompasses (6R)-6-(dimethylamino)-4,4-diphenyl-3-heptanone-(+)-N-protected glutamate salt (Formula V).

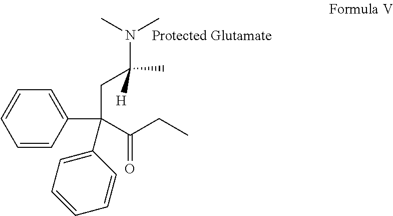

The N-protected group is preferably selected from mesyl chloride, tosyl chloride, acetyl chloride. The term ‘protected’ refers to messyl, tossyl, acetyl groups.

The process of the present invention to obtain (6R)-6-(dimethylamino)-4,4-diphenyl-3-heptanone free base is depicted in the reaction Scheme III.

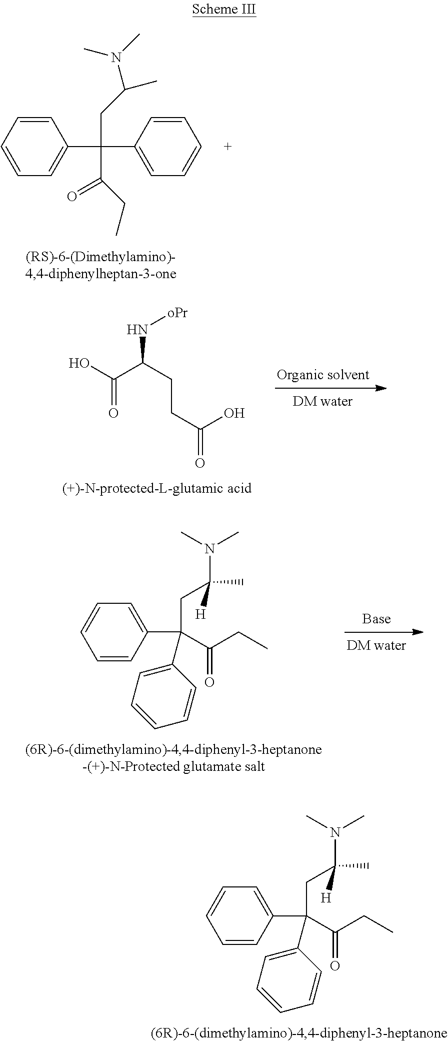

In another aspect the present invention discloses process for preparing (6R)-6-(dimethylamino)-4,4-diphenyl-3-heptanone hydrochloride salt from (6R)-6-(dimethylamino)-4,4-diphenyl-3-heptanone free base comprises:

Reacting (6R)-6-(dimethylamino)-4,4-diphenyl-3-heptanone free base with alcoholic hydrochloride in presence of alcoholic solvent by adjusting pH 1-1.5 at 25-30° C.;

Treating with charcoal and

Isolating (6R)-6-(dimethylamino)-4,4-diphenyl-3-heptanone hydrochloride salt.

The term alcoholic solvent refers methanol, ethanol, isopropanol, n-propanol, butanol. The alcoholic hydrochloride is selected from methanolic hydrochloric acid or Isopropanolic hydrochloride.

The following examples, which include preferred embodiments, will serve to illustrate the practice of this invention, it being understood that the particulars shown are by way of example and for purpose of illustrative discussion of preferred embodiments of the invention.

### EXAMPLES:

### Example 1

### Preparation of (+)-N-tosyl-L-glutamic Acid from L-glutamic Acid

To the 600 cc 2N sodium hydroxide solution was added 100 gm L-glutamic acid and the mixture was stirred for 10-15 mins at 25-30° C. Added slowly p-toluene sulfonyl chloride and heated to 70-75° C. Adjusted basic pH not less then 9 and stirred at 70-75° C. for 3-4 hrs. Cooled the reaction mass at 25-30° C. and pH adjusted to 1.5-2.0 using concentrated HCl. Charged ethyl acetate 1000 cc and stirred for 10-15 mins. Compound was extracted with 2×300 cc ethyl acetate from aqueous layer and the organic layer was washed with 2×300 cc water, 300 cc 5% sodium chloride solution. Distilled out solvent. Added 1000 ml n-Heptane and stirred for 1 hr. Filtered the solide & washed with 100 cc heptanes. Dried product at 40° C. for 1 hr and then at 55° C. for 3-4 hrs to obtain 76% (+)-N-tosyl-L-glutamic acid from L-glutamic acid.

### HPLC Purity: 98-99%

### Example 2

### Preparation of (RS)-6-(Dimethylamino)-4,4-diphenylheptan-3-One Free Base

To the 500 cc water added (RS)-6-(Dimethylamino)-4,4-diphenylheptan-3-one hydrochloride salt and stirred for 5-10 mins at 25-30° C. pH adjusted to 8.5-9.5 using aqueous ammonia and stirred for 1 h at 25-30° C. Filtered and washed with 100 cc water. The solid obtained was dried at 40-45° C. for 10-12 hrs to get 99% (RS)-6-(Dimethylamino)-4,4-diphenylheptan-3-one free base.

### HPLC Purity: 99.0-99.9%

### Example 3

### Preparation of (6R)-6-(dimethylamino)-4,4-diphenyl-3-heptanone-(+)-N-tosyl Glutamate Salt

To the 500 cc acetone added 100 g (RS)-6-(Dimethylamino)-4,4-diphenylheptan-3-one and stirred for 10-15 min at 25-30° C. Charged 58.3 g N-tosyl-1-glutamic acid and heated to 50-55° C. Added 15 cc water and stirred for 30 min at 50-55° C. Maintained the reaction mass for 12-15 hrs at 25-30° C. Cooled the reaction mass at 0-5° C. and stirred for 1 hr. Filtered and washed with 100 cc acetone. Charged 700 cc acetone to wet mass and stirred 30 min at 25-30° C. and heated to 50-55° C. Added 7.5 cc water and stirred for 30 min at 50-55° C. Maintained for 1 h at 25-30° C.; stirred 1 hr at 0-5° C., filtered and washed with 100 cc acetone. The solid obtained was dried at 40-45° C. for 3-4 hr to afford with yield of 66% (6R)-6-(dimethylamino)-4,4-diphenyl-3-heptanone- (+)-N-tosyl glutamate salt.

Chiral purity:L: 99.0-99.8% D: not more than 0.5%

### Example 4

### Preparation of (6R)-6-(dimethylamino)-4,4-diphenyl-3-heptanone-(+)-N-tosyl glutamate salt

To the 500 cc methyl ethyl ketone added 100g (RS)-6-(Dimethylamino)-4,4-diphenylheptan-3-one and stirred for 10-15 mins at 25-30° C. Charged 58.3 g (+)-N-tosyl-1-glutamic acid and heated to 50-55° C. Added 15 cc water and stirred for 30 mins at 50-55° C. Maintained reaction mass for 12-15 hrs at 25-30° C. Cooled reaction mass at 0-5° C. and stirred for 1 hr. Filtered and washed with 100 cc methyl ethyl ketone. Charged 700 cc methyl ethyl ketone to wet mass and stirred 30 min at 25-30° C. Heated to 50-55° C. Added 7.5 ml water and stirred for 30 mins at 50-55° C. Maintained for 1 h at 25-30° C. Stirred 1 hr at 0-5° C., filtered and washed with 100 cc methyl ethyl ketone. The solid obtained was dried at 40-45° C. for 3-4 hr to afford with yield of 66% (6R)-6-(dimethylamino)-4,4-diphenyl-3-heptanone-(+)-N-tosyl glutamate salt.

Chiral purity: L: 99.0-99.8% D: not more than 0.5%

### Example 5

### Preparation of (6R)-6-(dimethylamino)-4,4-diphenyl-3-heptanone free base

To the 700 cc water added 100 g (6R)-6-(dimethylamino)-4,4-diphenyl-3-heptanone-(+)-N-tosyl glutamate salt and stirred for 10-15 mins at 25-30° C. pH adjusted to 8.5-9.5 using aqueous ammonia and stirred for 1 hr at 25-30° C. Filtered and washed with 200 cc water. Solid obtained is dried at 40-45° C. for 10-12 hrs to obtain 99% (6R)-6-(dimethylamino)-4,4-diphenyl-3-heptanone free base.

HPLC purity: 99.0-99.9%

### Example 6

### Preparation of (6R)-6-(dimethylamino)-4,4-diphenyl-3-heptanone Hydrochloride salt

To the 400 ml Methanol added 100 g (6R)-6-(dimethylamino)-4,4-diphenyl-3-heptanone free base and stirred for 5-10 mins at 25-30° C. pH adjusted tot-1.5 by using 50-70 cc IPA HC1 at 25-30° C. Stirred reaction mass for 1 hr at 25-30° C. Added 5 g charcoal and stirred for 30 mins at 25-30° C. Filtered and washed with 100 cc methanol. Distilled out solvent and degased under vacuum at 45-50° C. for 10-15 mins Stripped out using 50 cc IPA and degased for 10-15 mins at 45-50° C. Added 300 ccIPA and stirred for 1 hr at 0-5° C. Filtered and washed with 100 cc chilled IPA. The solid obtained was dried at 40-50° C. for 10-12 hrs to get 93% (6R)-6-(dimethylamino)-4,4-diphenyl-3-heptanone Hydrochloride salt.

Chiral purity: L: 99.0 -99.9% D: not more than 0.5%

1. A novel method for preparing optically active (6R)-6-(dimethylamino)-4,4- diphenyl-3-heptanone (Formula I) comprising:
a) resolving a racemic mixture of (RS)-6-(Dimethylamino)-4,4-diphenylheptan-3-one by chemical reaction of (RS)-6-(Dimethylamino)-4,4-diphenylheptan-3-one with (+)-N-protected-L-glutamic acid in the presence of at least one organic solvent to obtain a solution of a (+)-N-protected-L-glutamate salt of (6R)-6-(dimethylamino)-4,4-diphenyl-3-heptanone; and
b) basifying the solution of the (+)-N-protected-L-glutamate salt of (6R)-6-(dimethylamino)-4,4-diphenyl-3-heptanone by adjusting a pH of the solution to between 8 and 12 to yield (6R)-6-(dimethylamino)-4,4-diphenyl-3-heptanone free base

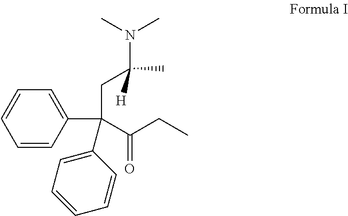 [SMILES: CCC(=O)C(C[C@@H](C)N(C)C)(c1ccccc1)c1ccccc1]

2. The process according to claim 1, wherein said at least one organic solvent comprises a ketone solvent.

3. The process according to claim 2, wherein the ketone solvent is selected from the group consisting of acetone, methyl ethyl ketone, diethyl ketone, methyl-tert- butyl ketone, isopropyl ketone, and mixtures thereof.

4. The process according to claim 1, wherein the pH of the solution is adjusted with a base selected from the group consisting of aqueous or alcoholic ammonia, carbonate salts or hydroxides of alkali and alkaline earth metals, sodium methoxide, sodium hydroxide, potassium hydroxide, calcium hydroxide, barium hydroxide, cesium hydroxide and lithium hydroxide, and mixtures thereof.

5. The process according to claim 1, wherein the reaction is carried out at a temperature of 40-60° C.

6. The process according to claim 1, wherein the enantiomeric purity of the (6R)-6-(dimethylamino)-4,4-diphenyl-3-heptanone free base is 99.0-99.9%.

7. An N-protected Glutamate salt of (6R)-6-(dimethylamino)-4,4-diphenyl-3- heptanone (Formula V)

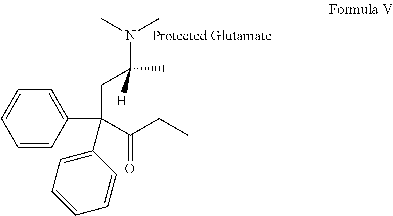

8. The N-protected Glutamate salt of (6R)-6-(dimethylamino)-4,4-dephenyl-3- heptanone (Formula V) according to claim 7, wherein the N-protected glutamate salt is protected with a protecting group selected from the group consisting of mesyl, tosyl and acetyl groups.

9. A process for preparing (6R)-6-(dimethylamino)-4,4-diphenyl-3-heptanone Hydrochloride salt comprising:
(a) preparing (6R)-6- (dimethylamino)-4,4-diphenyl-3-heptanone free base by the process of claim 1;
(b) reacting the (6R)-6-(dimethylamino)-4,4-diphenyl-3-heptanone free base with alcoholic hydrochloride in the presence of an alcoholic solvent by adjusting a pH of the solvent to between 1 and 1.5 at 25-30° C.;
(c) treating with charcoal and
(d) isolating the (6R)-6-(dimethylamino)-4,4-diphenyl-3-heptanone hydrochloride salt.

10. The process according to claim 9, wherein the alcoholic hydrochloride is selected from the group consisting of methanolic hydrochloric acid and isopropanolic Hydrochloride.

11. The process according to claim 9, wherein the alcoholic solvent is selected from the group consisting of methanol, ethanol, isopropanol, n-propanol and butanol.

12. The process according to claim 1, wherein the pH of the solution is adjusted with a base selected from the group consisting of potassium carbonate, potassium bicarbonate, sodium carbonate, sodium bicarbonate, cesium carbonate, calcium carbonate, and mixtures thereof.

13. The process according to claim 2, wherein the enantiomeric purity of the (6R)-6-(dimethylamino)-4,4-diphenyl-3-heptanone free base is 99.0-99.9%.

14. The process according to claim 3, wherein the enantiomeric purity of the (6R)-6-(dimethylamino)-4,4-diphenyl-3-heptanone free base is 99.0-99.9%.

15. The process according to claim 4, wherein the enantiomeric purity of the (6R)-6-(dimethylamino)-4,4-diphenyl-3-heptanone free base is 99.0-99.9%.
